(19) United States
(12) Patent Application Publication
(10) Pub. No.: US 2001/0001801 A1
(43) Pub. Date: May 24, 2001

(54) PROCESS FOR PRODUCING VITAMIN D3 AND PREVITAMIN D3

(76) Inventor: MONIKA JOHANNSEN, HAMBURG (DE)

Correspondence Address: MARK E WADDELL ESQ, BRYAN CAVE LLP, 245 PARK AVENUE, NEW YORK, NY, 101670034

(21) Appl. No.: 09/335,022

(22) Filed: Jun. 17, 1999

(30) Foreign Application Priority Data
Jun. 23, 1998 (EP) 98111490.3

(51) Int. Cl. C07J 53/00

(52) U.S. Cl. 552/500

(57) ABSTRACT

A process for the production of vitamin D₃ or previtamin D₃ from an isomer mixture comprises carrying out a separation by column chromatography using supercritical or liquid carbon dioxide, optionally with a modifier, as the mobile phase and an optionally modified silica gel as the stationary phase.

### SUMMARY

The invention relates to a process for the production of vitamin D₃ or previtamin D₃ from mixtures with other components, e.g., dehydrocholesterol, tachysterol and lumisterol, by column chromatography.

### BACKGROUND OF THE INVENTION

The D vitamins are biologically active substances that are essential for the regulation of calcium metabolism in higher animals. The various D vitamins differ by the nature of the side chain. The most important members in practice are vitamin D₂ (ergocalciferol) and vitamin D₃ (cholecalciferol). D previtamins are widely distributed in higher animals and plants. A sufficient photo-activation of previtamin D₃ occurs by UV irradiation. Historically, vitamin D₃ is also known as the anti-rickets vitamin. In our latitudes, rickets today is usually due not to previtamin deficiency, but to sunlight deficiency. Today, the industrial production of the D vitamins is carried out by the conversion of natural precursors, which are related to cholesterol.

Vitamin D₃ is insoluble in water, difficultly soluble in fatty oils and has good solubility in ethanol, chloroform, ether and acetone. Vitamin D₃ is sensitive to light, air, heat and acid. The melting point of vitamin D₃ lies in the range from 84 to 87° C. The solubility of D vitamins in supercritical or subcritical fluids, e.g. in supercritical CO₂ in the temperature range from 40 to 60° C. and a pressure range from 20 to 35 MPa, is known from the literature. The industrial process for the synthesis of vitamin D₃ is based on the irradiation of 7-dehydrocholesterol (DHC), which is produced from cholesterol. DHC is converted by irradiation into previtamin D₃ and this is isomerized to vitamin D₃ by gentle heating. Moreover, lumisterol and tachysterol are formed when DHC is irradiated. The yield of previtamin D₃ and consequently of vitamin D₃ depends essentially on the irradiation conditions.

Various processes are usual for the purification of the mother liquor at the conclusion of the irradiation. Thus, e.g., hitherto the undesired tachysterol has been converted using a Diels-Alder reaction into a tachysterol di-K salt adduct and the latter has subsequently been separated.

The conventional process has a number of disadvantages. The yield is limited by the state of equilibrium in the irradiation reaction. The performance of the Diels-Alder reaction requires additional chemicals and does not give a complete yield of vitamin D₃ or previtamin D₃ based on the crude product. Purification to crystalline grade requires additional reactions using chemicals such as pyridine and butyryl chloride, with again no complete reaction taking place. To sum up, therefore, there are losses of the valuable product.

The object of the invention is to avoid these disadvantages in the production of previtamin or vitamin D₃ from an isomer mixture of the kind formed, e.g., when using the irradiation process.

This is achieved in accordance with the invention by separating the vitamin or previtamin D₃ from the mixture by column chromatography.

Preferably, supercritical or liquid carbon dioxide with the addition of a polar modifier, e.g., ethanol, is used as the mobile phase and optionally modified silica gel is used as the stationary phase.

A preferred exemplified embodiment of the invention will be described hereinafter with reference to the accompanying drawings.

### BRIEF DESCRIPTION OF THE DRAWINGS

FIG. 1 is a flow diagram of the individual process steps.

FIG. 2 is a block diagram of the apparatus used.

### DETAILED DESCRIPTION OF THE INVENTION

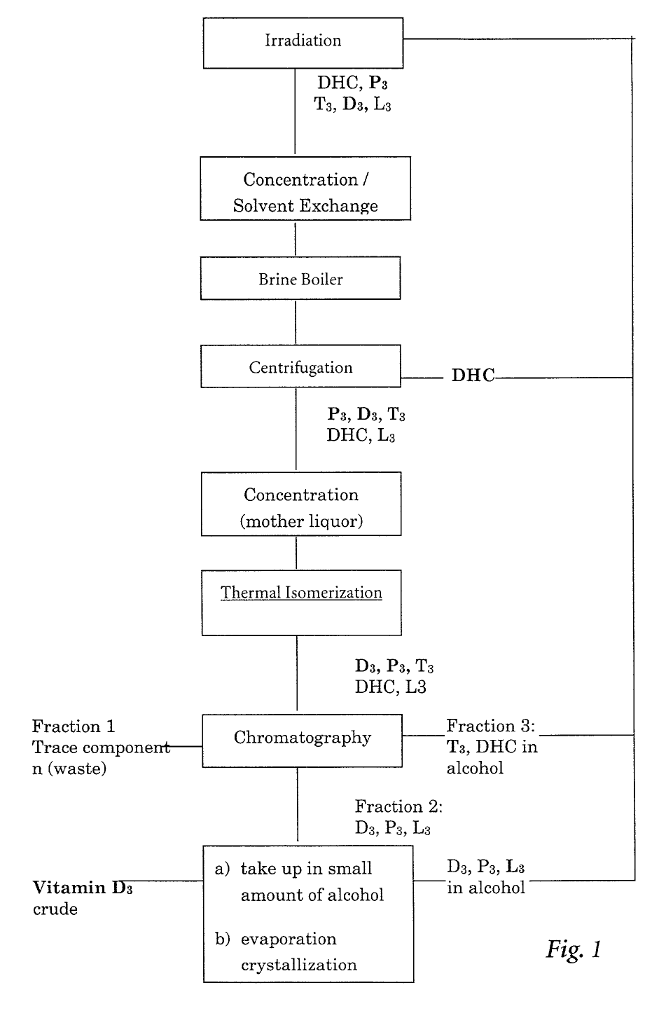

As set forth in FIG. 1, the mother liquor is firstly isomerized thermally, then chromatographed. The remaining 7-dehydrocholesterol (DHC) as well as the tachysterol (T₃) are removed and recycled to the irradiation batch. Since the photochemical reaction is an equilibrium reaction, the recycling of the actual undesired components prevents the renewed formation of these, so that the yield is increased. Vitamin D₃ can be crystallized from the resulting useful fraction (fraction 2). The proportion of the vitamin D₃, previtamin D₃ (P₃) and lumisterol (L₃) remaining in solution is likewise recycled to the irradiation batch. If desired, fraction 2 can be additionally separated by chromatography.

A chromatographic process gives the following advantages:

avoidance of the Diels-Alder reaction,

byproduct fractions are recycled into the process,

higher yield, and

purer product;

and especially when using SFC (chromatography with supercritical gases):

a solvent-free process step,

simple separation by pressure release and

problem-free circulation of the eluent.

In principle, the process in accordance with the invention is carried out by combining the isomer mixture, already under pressure if desired, with the supercritical or liquid mobile phase, applying the whole, optionally followed by more mobile phase, to the chromatography column packed with the aforementioned stationary phase and then allowing it to flow through (elute). The elution being effected under the chosen temperature and pressure conditions and, on the basis of the strong interactions between the stationary phase and the individual components of the mixture, these components being separated per unit of time. Being eluted in succession from the column, the components dissolved in the mobile phase (eluates) after sequentially detection (determined), being collected in receivers, are determined by the detection agent and the carbon dioxide being removed from the collected material by decompression (volatization) so that finally the resulting separated components or “fractions” (inter alia the desired vitamin D₃ or previtamin D₃) are free from carbon dioxide in the individual receivers. If desired, after the elution and exit from column, the eluate can be subjected to one or more additional similar chromatographic procedures in order to achieve an even better separation of the components. The same applies to any particular fraction which does not having the desired purity.

Any suitable mixture that contains vitamin D₃ or previtamin D₃ can be used as the mixture of vitamin D₃ isomers in the process in accordance with the invention. Thus, for example, a synthetic mixture can be used before or after thermal isomerization.

The isomer mixture containing vitamin D₃ and/or previtamin D₃ is normally applied without dilution together with the supercritical or liquid mobile phase to the chromatography column packed with the stationary phase used in accordance with the invention, although it can previously be dissolved in a suitable solvent, e.g. a lower alkanol, preferably ethanol. Preferably, however, the mixture is used without dilution.

The supercritical carbon dioxide used in the process in accordance with the invention is in the form of carbon dioxide which is held at a temperature of at least about 31° C. and a pressure of at least about 7.3 MPa and is neither completely liquid nor completely gaseous but is a hybrid of the two physical forms. The liquid carbon dioxide, which is used as an alternative in the process in accordance with the invention, has a temperature of less than about 31° C. and a pressure that lies above about 7.3 MPa. The advantages of using carbon dioxide are its non-toxicity, non-flammability and easy removal by decompression of the collected eluates, without leaving a potentially harmful residue in the separated fractions, e.g., vitamin D₃ or previtamin D₃. Further, very pure carbon dioxide is widely available and inexpensive and, if desired, can be used with an organic co-solvent (modifier), e.g., the already mentioned ethanol or with other alkanols, e.g., methanol, or alkanes, e.g., n-hexane, or ketones e.g., acetone, as part of the mobile phase. Since the critical temperature of carbon dioxide is not much higher than room temperature and the substances to be obtained in accordance with the invention are temperature-sensitive (thermolabile), carbon dioxide is advantageously suited as the mobile phase in the process in accordance with the invention.

The modified silica gel used as the stationary phase in the process in accordance with the invention is advantageously present in the form of substantially homogeneous, packed, non-uniform or preferably spherical particles with a particle size of about 5 to 25 mm. ZORBAX and HYPERPREP are examples of commercially available silica gels. The former has a specific surface area S_{BET} of 350 m²/g, a pore volume Vₚ of 0.53 ml/g, a pore diameter D of 60-150 Å and a particle size dp₅₀ of 10 μm, whereas the latter has an S_{BET} of 300 m²/g, a Vₚ of 0.7 ml/g, a D of 100 Å and a particle size dp₅₀ of 12 μm.

In order to keep the carbon dioxide used as the mobile phase in the process in accordance with the invention in the supercritical or liquid range, certain temperature and pressure conditions must be maintained, not only when introducing the carbon dioxide into the chromatography column packed with the stationary phase but also during the subsequent elution. The process is conveniently carried out at in the temperature range from 0° C. to about 100° C. and at a pressure of about 7.5 MPa to about 32.0 MPa. Preferably, the temperature range is from about 30° C. to about 60° C. and the respective pressure range is 7.5 to 15.0 MPa. The density of carbon dioxide can be adjusted via the pressure and temperature and in the last-mentioned temperature and pressure range is from about 170 kg/m³ to about 850 kg/m³.

Not only the temperature conditions and the pressure conditions under which the process in accordance with the invention is carried out, but also the choice of the stationary phase and the mobile phase, exert an influence on the separation result. In general, a temperature increase or pressure reduction moves the various eluted isomers apart in time, whereas a pressure increase or temperature reduction draws the eluates together, so that the optional variation of these parameters can determine the course of the process in accordance with the invention per unit time.

Optional influence of different mixtures of the mobile phase.

The detection of the components dissolved in carbon dioxide successively eluted on the chromatographic column (eluates) is effected in parallel, preferably by a UV detector and a flame ionization detector (FID). Detection is a way of electronically controlling the distribution of the various eluates among the receivers. Such technology is known per se, as is the method of removing carbon dioxide (by decompression) from the respective collected material.

The invention is illustrated on the basis of the following Example.

### EXAMPLE

An apparatus from the Hewlett Packard company (HP G1205A SFC) is used for the investigation of the chromatographic production of the components, particularly of vitamin D₃ or previtamin D₃, from an isomer mixture. The apparatus consists of the basic units comprising pump, oven with gas phase detector, external detector and automatic sampler. A flow diagram of the apparatus is shown in FIG. 3. The apparatus is supplied continuously with liquid carbon dioxide. Depending on the chosen pressure and temperature conditions, the mobile phase can be operated in the supercritical range (above about 31° C. and 7.3 MPa in the case of pure CO₂) or in the subcritical range.

The apparatus was operated in the “downstream” mode. This operational procedure signifies that when packed columns having an internal diameter greater than 1 mm are used the column back-pressure in the system is used as a fixed regulator in addition to the flow. The feeding of the pump with liquid high-pressure CO₂ (P>>35 MPa) is effected via the in-house gas network. The pump inlet pressure is reduced to Pᵢₙₚᵤₜ>>10 MPa using a pressure reducer. This setting can be varied within certain limits, thereby ensuring that the pump is supplied with liquid phase. The delivery and compression up to the required column pre-pressure is effected using a piston pump. The pump head has a temperature of 278 K. in order to dissipate the resulting heat of compression. The analytical column is situated in the oven in which the mobile phase is heated to the test temperature. The sample introduction is effected by a pneumatically controlled four-way Rheodyne valve that is equipped with a 5 μl internal loop. The automatic sample deliverer, which is equipped with a 50 μl syringe, fills the internal loop with sample solution via the injection port. The sample then travels with the mobile phase to the column. Here a separation of the mixture takes place on the basis of differences in the strength of interaction between the stationary phase and the individual components of the sample solution. The components of the mixture (in the ideal case) are eluted successively from the column. After the analytical column the stream of eluent undergoes a permanent split. This split is achieved by a fixed restrictor which conducts the split stream to the gas phase detector, a flame ionization detector (FID). The larger part of the stream of eluent remaining after the split passes the a diode array detector (DAD). A SFC decompression unit is connected after the DAD.

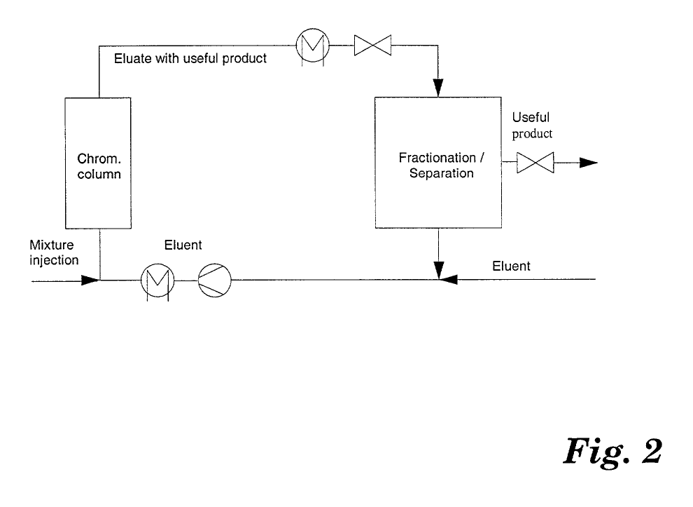

The chromatograms are recorded with the data system. The tests by analytical SFC show successful separation of the vitamin D₃ isomers. Separation with CO₂ as the eluent without a modifier is not possible in this case. On the other hand, excellent results are obtained by the addition of small amounts of alcohol to the CO₂. A variety of normal phase materials based on silica are used as the stationary phase. The selectivity between vitamin D₃ and tachysterol, e.g., on a cyano phase, lies at about a >>1.5 (see FIG. 2), which is optimal for preparative separation. The nature of the alcohol (methanol, ethanol, iso-propanol) has practically no influence. The selectivity increases with simultaneous retention time prolongation the smaller the proportion of modifier. The retention time can be shortened to a certain extent by increasing the density of the CO₂ (or of the mobile phase).

These and other objects are included within the scope of the claim invention

What is claimed is:

1. A process for the production of vitamin D₃ or previtamin D₃ from mixtures with other components, e.g., dehydrocholesterol, lumisterol and tachysterol, which process comprises separating the vitamin D₃ or previtamin D₃ by column chromatography.

2. A process according to claim 1, wherein carbon dioxide, which is selected from the group consisting of supercritical carbon dioxide and liquid carbon dioxide, and a modifier are used as the mobile phase.

3. A process according to claim 1, wherein a silica gel is used as the stationary phase.

4. A process according to claim 1, wherein the silica gel is in the form of homogeneously packed, spherical particles having a particle size of about 5 to 25 μm.

5. A process according to claim 1, wherein a reaction mixture synthetically produced by irradiation is used as a mixture of vitamin D₃ isomers.

6. A process according to claim 1, which is carried out in the temperature range from about 30° C. to about 60° C. and in the pressure range from about 7.0 to about 15.0 MPa.
